(19) United States
(12) Patent Application Publication
(10) Pub. No.: US 2013/0295055 A1
(43) Pub. Date: Nov. 7, 2013

(54) PROBIOTIC COMPOSITION FOR LIVESTOCK, AND METHOD FOR PREPARING SAME

(71) Applicants: Day Jung, Gwangju-si (KR); Yun Jeong Heo, Seoul (KR); Changsun Choi, Anyang-si (KR); Dae Keon Choi, Seoul (KR)

(72) Inventors: Day Jung, Gwangju-si (KR); Yun Jeong Heo, Seoul (KR); Changsun Choi, Anyang-si (KR); Dae Keon Choi, Seoul (KR)

(73) Assignees: CTC BIO, INC., Seoul (KR); GWANGJU AGRICULTURAL TECHNOLOGY CENTER, Gwangju-si, Gyeonggi-do (KR)

(21) Appl. No.: 13/978,593

(22) PCT Filed: Jan. 10, 2012
(86) PCT No.: PCT/KR12/00242

§ 371 Date: Jul. 8, 2013

(30) Foreign Application Priority Data
Jan. 11, 2011 (KR) 10-2011-0002613

(51) Int. Cl. A61K 35/74

(52) U.S. Cl. CPC A61K 35/741
USPC 424/93.4

(57) ABSTRACT

The present invention relates to an eco-friendly probiotic composition, and more particularly, to a probiotic composition for livestock production, comprising a lactic acid bacteria culture medium and an organic acid, wherein the organic acid is contained in an amount of 0.002 to 0.1 parts by weight relative to 1 parts by weight of the lactic acid bacteria culture medium. The present invention also relates to a method for preparing the composition. The eco-friendly probiotic composition of the present invention does not contain antibiotics and may exhibit complex effect of sterilization and virucidal effectiveness.

### TECHNICAL FIELD

The present invention relates to a probiotic composition for livestock production and a method for preparing the same, and more particularly to a probiotic composition for livestock production, comprising a lactic acid bacteria culture medium (broth) and an organic acid.

The present invention also relates to a method for improving a preservative effect of the probiotic composition for livestock production.

### BACKGROUND ART

In the conventional field of livestock production, antibiotics, synthetic antimicrobials, etc. have been widely used for improving livestock productivity.

However, antibiotics residue in animal products have recently mentioned as a social issue, and following concerns that the excessive use of antibiotics for animals may account for the appearance of superbacteria due to acquisition of resistance by pathogenic bacteria, advanced countries such as the United States, and Europe tighten rules and management on antibiotics use. Following this trend, attempts to minimize chemicals usage and maximize growth performance of animals have recently been to practical use by using probiotics, the microorganism preparations which express competitive exclusion effect against pathogenic microorganisms, and intensify disease-resistant properties of animals through suppressive action of metabolites.

Probiotics, the preparations containing live beneficial microorganisms, refer to preparations of microorganisms that settle down in the animal intestine, inhibit the growth of harmful microorganisms through competitive exclusion against other pathogenic microorganisms, and stimulate animal growth and improve feed efficiency, by helping digestion and absorption of ingested feed, and are also called direct-fed microbials (DFM). Among microorganisms being used as live microbes for these probiotics, lactic acid bacteria (LAB) which have excellent immunity-enhancing, feedlot environment improving, and animal productivity-enhancing effects are the most frequently used as a safe microbe that does not give any specific pathogenicity to animals or humans. Utilizing probiotics using lactic acid bacteria becomes an effective alternative to overcome factors such as breeding management intensification due to the recent size expansion of livestock industry, high potency feeding-induced stress of digestive organs and lack of exercise, decrease in useful environmental microorganisms due to disinfection and the like, and other factors which may diminish animal productivity.

In addition, the most frequently used probiotic, lactic acid bacteria shows a variety of anti-bacterial activities which are considered to result from various kinds of organic acids, hydrogen peroxide, bacteriocins, and the like. In particular, for the recently attention-attracting antibacterial substance bacteriocin among them, it is known that its cell wall permeability against harmful strains increases at low pH, and thus, it's sterilizing effectiveness increases. Therefore, it is considered that the antibacterial effectiveness of lactic acid bacteria can be increased by lowering the pH of lactic acid bacteria culture medium.

Meanwhile, regarding probiotics for livestock production, research on probiotics for livestock production having usefulness other than native probiotic effectiveness of probiotics has been partially proceeded. In the case of animal products, regarding food poisoning bacteria such as Salmonella, Escherichia coli (E. coli) which are propagated through excreta, a foot-and-mouth disease virus which has recently resulted in large economic and social damage in South Korea, and a highly pathogenic avian influenza virus which can be propagated by migratory birds, and thus, cause troubles every year, research on probiotics for livestock production which use probiotics for livestock production as well as individual therapeutic agents or prophylactic agents, and thus, can exert complex effectiveness is unsatisfactory. It is difficult to simply use only antiseptics to control food poisoning bacteria, including Salmonella, a foot-and-mouth disease virus, an avian influenza virus, and the like in feedlot interior sanitary control. Environmental pollution generated by regular use of antiseptics, decline in animal immunity due to regular exposure to antiseptics, and the like also cause troubles in the field of livestock production. There is an urgent need for developing probiotics which may exert complex effectiveness that resolves adverse effect and residue issues resulting from abuse of harmful germicides and antiseptics for the purpose of pathogen controls, and concurrently reduces animal diseases resulting from proliferation of harmful microorganisms to a minimum. Probiotics can function beneficially to animal productivity through regular feeding, and at the same time, exert their effectiveness for inhibiting harmful microorganisms via application in feedlots.

Therefore, development of probiotic compositions for livestock production having complex effectiveness, which are economical, easy to use, and have no concern for adverse effects are urgently needed, in addition to individual therapeutic agents or prophylactic agents, related to food poisoning bacteria, malignant livestock infectious diseases such as foot-and-mouth disease, avian influenza virus, and the like in animal products.

### DISCLOSURE OF THE INVENTION

### Technical Problem

Therefore, the present invention is to provide a new probiotic composition for livestock production, provided with disinfection effectiveness against food poisoning bacteria and the like as well as probiotic effectiveness, by the addition of an organic acid, as a probiotic composition for livestock production comprising lactic acid bacteria as major live bacteria.

The present invention is also to provide an optimal condition for a method of adding an organic acid to a probiotic composition for livestock production comprising lactic acid bacteria as major live bacteria, in order to combine the effectiveness of sterilizing bacteria such as food poisoning bacteria.

Further, the present invention is to provide a virucidal probiotic composition for livestock production, provided with disinfection effectiveness against viruses, as a probiotic composition for livestock production comprising lactic acid bacteria as major live bacteria.

The present invention is also to provide a method for improving a preservative effect of a probiotic composition for livestock production, characterized in that a liquid stabilizer is added to improve a preservative effect of the probiotic composition.

### Technical Solution

Therefore, the present invention provides a probiotic composition for livestock production, comprising a lactic acid bacteria culture medium and an organic acid, wherein the organic acid is contained in an amount of 0.002 to 0.1 parts by weight relative to 1 parts by weight of the lactic acid bacteria culture medium.

The present invention also provides a method for preparing a probiotic composition for livestock production, comprising the steps of (a) preparing a lactic acid bacteria culture medium; and (b) preparing a probiotic composition of a pH of 2.1 to 3.5 by adding an organic acid to the above (a) lactic acid bacteria culture medium.

Further, the present invention provides a method for improving a preservative effect of a probiotic composition for livestock production, characterized in that a liquid stabilizer is added to a probiotic composition for livestock production, comprising a lactic acid bacteria culture medium and an organic acid, wherein the organic acid is contained in an amount of 0.002 to 0.1 parts by weight relative to 1 parts by weight of the lactic acid bacteria culture medium.

### Advantageous Effects

According to the present invention, an economical, safe, and new probiotic composition for livestock production, wherein an organic acid is added to lactic acid bacteria as major live bacteria, and thereby, complex effectiveness is provided, can be provided.

Also, the present invention can provide a probiotic composition for livestock production, provided with the effectiveness of sterilizing bacteria such as food poisoning bacteria as well as probiotic effectiveness, by the addition of an organic acid to lactic acid bacteria.

According to another aspect of the present invention, an optimal condition for preparing a new probiotic composition for livestock production, wherein an organic acid is added to lactic acid bacteria as major live bacteria, can be provided.

Also, the present invention can provide a probiotic composition for livestock production, provided with the disinfection effect against viruses, by the addition of an organic acid to lactic acid bacteria.

Further, the present invention provides a method for improving a preservative effect of a probiotic composition for livestock production, and thus, can improve viability of lactic acid bacteria in a probiotic composition for livestock production.

### BRIEF DESCRIPTION OF THE DRAWINGS

FIG. 1 is a graph showing a pH change depending on the incubation time of lactic acid bacteria culture medium according to an embodiment of the present invention;

FIG. 2 is photographs showing the results of incubation tests following the examination for the number of lactic acid bacteria of Comparative Example 1 and Examples 1 to 6;

FIG. 3 is a graph showing the results of evaluation for Salmonella control effect of Comparative Example 1 and Examples 5 and 6 of the present invention;

FIG. 4A shows the results of evaluation for Salmonella control effect by adding lactic acid bacteria culture medium and citric acid to Salmonella, and FIG. 4B shows the results of evaluation for Salmonella control effect by adding citric acid alone to Salmonella;

FIG. 5A shows the results of evaluation for Salmonella control effect by adding lactic acid bacteria culture medium (broth) and lactic acid to Salmonella, and FIG. 5B shows the results of evaluation for Salmonella control effect by adding lactic acid alone to Salmonella;

FIG. 6A shows the results of evaluation for Salmonella control effect by adding lactic acid bacteria culture medium and malic acid to Salmonella, and FIG. 6B shows the results of evaluation for Salmonella control effect by adding malic acid alone to Salmonella;

FIG. 7A shows the results of evaluation for E. coli control effect by adding lactic acid bacteria culture medium and citric acid to E. coli, and FIG. 7B shows the results of evaluation for E. coli control effect by adding citric acid alone to E. coli;

FIG. 8A shows the results of evaluation for E. coli control effect by adding lactic acid bacteria culture medium and lactic acid to E. coli, and FIG. 8B shows the results of evaluation for E. coli control effect by adding lactic acid alone to E. coli;

FIG. 9A shows the results of evaluation for E. coli control effect by adding lactic acid bacteria culture medium and malic acid to E. coli, and FIG. 9B shows the results of evaluation for E. coli control effect by adding malic acid alone to E. coli;

FIG. 10A shows the results of evaluation for Listeria control effect by adding lactic acid bacteria culture medium and citric acid to Listeria, and FIG. 10B shows the results of evaluation for Listeria control effect by adding citric acid alone to E. coli;

FIG. 11A shows the results of evaluation for Listeria control effect by adding lactic acid bacteria culture medium and lactic acid to Listeria, and FIG. 11B shows the results of evaluation for Listeria control effect by adding lactic acid alone to Listeria; and

FIG. 12A shows the results of evaluation for Listeria control effect by adding lactic acid bacteria culture medium and malic acid to Listeria, and FIG. 12B shows the results of evaluation for Listeria control effect by adding malic acid alone to Listeria.

### MODE FOR CARRYING OUT THE INVENTION

[National Research and Development Program that supported the Invention]

[Project Number] K111101

[Ministry] Gyeonggi-do

[Research Management Specialized Institution] Gyeonggi Institute of Science and Technology Promotion

[Research Project Name] Public Technology Development

[Project title] Development of Lactic Acid Bacteria-Organic Acid Complex Feed Additive For Prevention of Foot-and-mouth Disease

[Contribution Ratio] 1/1

[Institute] Gwangju Agricultural Technology Center

[Project period] 2011 Jun. 1-2012 May 31

Hereinafter, the present invention will be described in more detail with reference to preferable embodiments. Prior to the description, terms or words used in the present specification and claims should not be construed as limited to general and dictionary meaning, but should be construed based on the meanings and concepts according to the spirit of the present invention, on the basis of the principle that the inventor is permitted to define concepts of terms appropriately for the best explanation for his invention. Therefore, the constructions of embodiments described in the specification are just most preferable embodiments of the present invention and do not represent all the spirit of the present invention, and thus, it should be understood that various equivalents and modifications which can replace them may be present at the time of application of the present invention.

The present invention is characterized in that a probiotic composition for livestock production comprises lactic acid bacteria culture medium and an organic acid.

The lactic acid bacteria are the most representative probiotic among microorganisms used as probiotics, and since they do not cause specific pathogenicity to animals or humans, they are the most effective and safest bacteria to be used as a probiotic except in special cases. These lactic acid bacteria use sugar, the energy source, and produce lactic acid as a fermentation product, and when used in animal products, they inhibit growth of putrefactive or pathogenic microbes, and thus, have intestinal function-strengthening and digestive disease-preventing effects. In addition, they produce digestive enzymes and thus increase feed efficiency, enhance weight gain of animal products, and inhibit putrefactive or food poisoning bacteria, and thereby reduce bad smell in livestock sheds.

Meanwhile, the foot-and-mouth disease virus which has become an object of public concern is the most active at a pH of 7.2 to 7.6, and can survive even at a pH of 6.7 or less, or a pH of 9.5 or more, at 4° C. or less, for any substantial length of time. However, it is known to die rapidly at a pH of 5.0 or less, or a pH of 11.0 or more, and be destroyed completely at 56° C. for about 30 minutes. Avian influenza virus is also very sensitive to acidity like the foot-and-mouth disease virus, and dies rapidly at a pH of 5.0 or less. Therefore, if lactic acid bacteria are used in large quantities for foot-and-mouth disease, avian influenza-related livestock burial sites, they can self-reproduce, inhibit penetration of putrefactive or food poisoning bacteria, prevent bad smell from the burial sites, and result in some effects like reduced gas generation. Generally, when these lactic acid bacteria are used as probiotics for livestock production in practice, a dilution step is used, and then, the probiotic effectiveness may be maintained due to an excessive pH increase, depending on lactic acid bacteria incubation and dilution concentrations, but it is not easy for lactic acid bacteria to take the above effects.

Therefore, the present inventors have studied probiotic compositions for livestock production, which can have complex effectiveness, such as virucidal effectiveness, sterilization effectiveness, including probiotic effectiveness, in the administration or application of probiotic compositions for livestock production to animal products, and found that the addition of an organic acid to lactic acid bacteria which show the most excellent probiotic effectiveness can exhibit complex effectiveness such as virucidal effectiveness on foot-and-mouth disease and avian influenza, and sterilization effectiveness.

Lactic acid bacteria used for the probiotic composition for livestock production according to the present invention are classified into species—cocci such as Enterococcus, bacilli such as Lactobacillus, and an amorphous species such as Bifidobacteria. In the present invention, bacteria which have an excellent ability to kill harmful bacteria and have a high survival rate even at low acidity of 3.0 or less may be used without limitation, and Lactobacillus plantarum, Lactobacillus casei, Lactobacillus acidophilus, Lactobacillus reuteri, Lactobacillus delbrueckii, Lactobacillus brevis may be used.

The lactic acid bacteria culture medium is not particularly limited, but the pH of the lactic acid bacteria culture medium may be 4.0 or less, considering an optimal pH for showing complex effectiveness such as probiotic effectiveness and disinfection effectiveness after an organic acid addition and dilution.

The organic acid is added, in order to control an excessive pH increase induced by dilution when the lactic acid bacteria culture medium is administered or applied, to maintain probiotic effectiveness of the probiotic composition, and to show sterilization effectiveness of the probiotic composition. The organic acid may be contained in an amount of 0.002 to 0.1 parts by weight, and more preferably, 0.01 to 0.02 parts by weight, relative to 1 parts by weight of the lactic acid bacteria culture medium. Where the amount of the organic acid is less than 0.02 parts by weight, the sterilization effectiveness may be insufficient due to an excessive pH increase induced by dilution when the lactic acid bacteria culture medium is administered to animals or applied to livestock sheds. Where the amount of the organic acid is more than 0.1 parts by weight, the sterilization effectiveness increases, but, the growth of lactic acid bacteria is inhibited by an excessive addition, and the probiotic effectiveness may decrease. In addition, the pH of the probiotic composition in accordance with the addition of the above amounts of the organic acid may be 2.1 to 3.5. The organic acid is not particularly limited, but for safety assurance, it may be one selected from the group consisting of citric acid anhydrous, malic acid, and lactic acid for food additives.

Here, the probiotic composition comprising the lactic acid bacteria culture medium and the organic acid is diluted for administration to animals or application to livestock sheds. For the probiotic effectiveness, the probiotic composition may be one which is diluted 10 to 100 times and of which a pH is 4 or less.

It was found that this probiotic composition according to the present invention maintained the probiotic effectiveness equal to other probiotic compositions using conventional lactic acid bacteria as probiotics, and furthermore, it showed the virucidal effectiveness on the foot-and-mouth disease and avian influenza.

Meanwhile, examples of livestock in which the above effectiveness of the probiotic composition for livestock production according to the present invention can be exhibited include cattle, pigs, goats, and the like, but the present invention is not limited thereto.

Hereinafter, a method for preparing a probiotic composition for livestock production according to the present invention will be described in detail.

A method for preparing a probiotic composition for livestock production according to the present invention comprises the steps of (a) preparing a lactic acid bacteria culture medium; and (b) preparing a probiotic composition of a pH of 2.1 to 3.5 by adding an organic acid to the above (a) lactic acid bacteria culture medium.

The lactic acid bacteria which are included in the lactic acid bacteria culture medium are not particularly limited, but may be lactobacillus which has an excellent ability to kill harmful bacteria. For this, bacteria which are appropriate for surviving at pHs to have an ability to kill harmful bacteria and exhibit sterilization and virucidal effectiveness were selected and used in the present invention, and any lactic acid bacteria with strong acid resistance may be used without limitation for the purposes of the present invention.

In addition, the medium used for preparation of the lactic acid bacteria culture medium may be a medium which is generally used, but conventional lactic acid culture methods are not preferred for unit cost of culture.

Accordingly, the lactic acid bacteria culture medium used for the method for preparing the probiotic composition for livestock production according to the present invention may be a culture medium for lactic acid bacteria, comprising 5 to 50% of yeast extract, 1 to 10% of glucose, and 1 to 10% of sucrose, and provides an economical culture medium for lactic acid bacteria. The culture medium for lactic acid bacteria which can be used in the present invention uses vegetable raw materials only, and shows a greatly excellent activity of 2.1×10⁸ lactic acid bacteria upon incubation, compared to 1.0×10⁷ lactic acid bacteria by the conventionally used culture medium. In addition, the unit cost of culture can be reduced to a level of ⅓.

The probiotic composition may be prepared by using the culture medium for lactic acid bacteria like this, performing a stationary culture at 34 to 36° C. for 24 to 48 hours. Where the above temperature and time conditions are not fulfilled, the sterilization effectiveness may be insufficient upon dilution. Most preferably, the above temperature and times conditions, and a pH of 4 or less of cultured lactic acid culture medium may be applied for the sterilization effectiveness.

In addition, after completion of lactic acid bacteria culture, 0.05 to 0.15% by weight of glutamic acid and 0.5 to 1.5% by weight of sodium chloride (NaCl) may be added relative to the lactic acid bacteria culture medium, as additives for freeze protection in winter.

The organic acid may be diluted 10 to 100 times, relative to 1 parts by weight of the lactic acid bacteria culture medium, and added to the lactic acid bacteria culture medium, and thus, a pH of the probiotic composition may be 2.1 to 3.5. A kind of the organic acid used is not particularly limited, but for safety assurance, it may be one selected from the group consisting of citric acid anhydrous, malic acid, and lactic acid for food additives. Meanwhile, in accordance with one aspect of the probiotic composition for livestock production according to the present invention, dilution with distilled water and so on is finally done for administration to animals or application to livestock sheds. Here, it is important to maintain an appropriate pH (preferably a pH of 4 or less) for exhibition of probiotic effectiveness, regardless dilution. In the present invention, it was confirmed that after the addition of the organic acid to the lactic acid bacteria culture medium, 10 times to 100 times dilution with distilled water and the like resulted in the probiotic composition, of which a pH was maintained at 4 or less and which showed an effective sterilization effect. In addition, the present invention relates to the virucidal effect of the probiotic composition for livestock production, and examples of the virus may include all of animal viruses which may be generated from livestock sheds, and may be died generally at low pH, such as influenza virus, foot-and-mouth disease virus, swine vesicular disease virus, avian influenza virus, and the like.

The probiotic composition for livestock production of the present invention may be diluted 1 to 100 times, preferably 10 to 50 times to use, for the effective exhibition of virucidal effect of the probiotic composition for livestock production.

Also, the present invention relates to a method for improving a preservative effect of a probiotic composition for livestock production, characterized in that a liquid stabilizer is added to a probiotic composition for livestock production, comprising a lactic acid bacteria culture medium and an organic acid, wherein the organic acid is contained in an amount of 0.002 to 0.1 parts by weight relative to 1 parts by weight of the lactic acid bacteria culture medium.

The liquid stabilizer of the present invention may be selected from the group consisting of trehalose, glycerol, fructooligosaccharide, betaine, proline, dextran, maltose, sucrose, mannitol, polyol, silica gel, aminoguanidine, pyridoxamine, and a mixture thereof, and preferably, may be glycerol or fructooligosaccharide. The liquid stabilizer may be added in an amount of 0.001 to 0.1 parts by weight relative to 1 parts by weight of the lactic acid bacteria culture medium, and the addition of the liquid stabilizer can increase survivability of the liquid lactic acid bacteria of the present invention.

The probiotic composition for livestock production of the present invention may be prepared in a liquid or powder form. To formulate it in a powder preparation, known methods in the art, such as using lactic acid bacteria perlite or alginate may be used.

The probiotic composition for livestock production prepared may be added to feeds and administered, or directly orally administered to use, and may be applied to livestock sheds to disinfect viruses and bacteria in the livestock sheds.

Hereinafter, the present invention will be described in detail with reference to embodiments. However, the following embodiments are for illustrative purposes only, and the contents of the present invention are not limited by the following embodiments.

1. Evaluation of Sterilization Effectiveness of Probiotic for Livestock Production

1.1 Preparation of Lactic Acid Bacteria Culture Medium

To select lactic acid bacteria strain having a sufficient acid resistance suitable for the purpose of the present invention, the following separation method was carried out.

10 samples of Kimchi and 10 samples of poultry manure were diluted 10 times with 0.85% saline, and then, 2% of citric acid was added thereto, and stored at 4° C. for 48 hours. Subsequently, 0.1 mL of each sample was smeared using a glass rod onto MRS agar medium (Difco) plates. Subsequently, plates were incubated in a constant temperature incubator at 35° C. for 36 hours. The produced each colony was streak-inoculated onto MRS agar medium plates, and incubated in a constant temperature incubator at 35° C. for 36 hours to isolate lactic acid bacteria colonies. The isolated each lactic acid bacteria colony was incubated in a constant temperature incubator at 35° C. for 24 hours to culture final lactic acid bacteria, and then, the pH of the MRS medium was examined to select lactic acid bacteria of a pH of 3.8 or less only.

According to conventional methods known in the art, sequence analysis of 16S rDNAs of the selected lactic acid bacteria was carried out. Consequently, the isolated lactic acid bacteria by the above method were identified as Lactobacillus plantarum, Lactobacillus casei, Lactobacillus acidophilus, Lactobacillus reuteri, Lactobacillus delbrueckii, and Lactobacillus brevis. Therefore, the above bacteria were identified as strains having a sufficient acid resistance, and it was confirmed that those bacteria can be used for the acidity-controlled, probiotic composition for livestock production of the present invention.

To perform more specific experiments, Lactobacillus plantarum was selected among the above bacteria. In the subsequent experiments, Lactobacillus plantarum were used.

To inoculate Lactobacillus plantarum mixture at a level of 2.0×10⁸ as a 1% starter, lactic acid culture medium was prepared by using the following medium. For medium preparation, 2.5% by weight of yeast extract, 1.5% by weight of glucose, and 2% by weight of sucrose were included. Then, stationary culture was performed at 35° C. for 48 hours to prepare the lactic acid culture medium. In addition, as additives for freeze protection, 0.1% by weight of glutamic acid and 1% by weight of sodium chloride were added to the lactic acid bacteria culture medium.

1.2 Dilution and Addition of Organic Acid

(1) Citric Acid Addition to Lactic Acid Bacteria Culture Medium

After the lactic acid bacteria incubation, citric acid anhydrous for a food additive was diluted at dilution rates described in the following Table 1, and added to the lactic acid bacteria culture medium, for citric acid to be included in an amount of 0.002 parts by weight, 0.0025 parts by weight, 0.0033 parts by weight, 0.005 parts by weight, 0.01 parts by weight, 0.02 parts by weight, 0.03 parts by weight, 0.05 parts by weight, 0.07 parts by weight, and 0.1 parts by weight relative to 1 parts by weight of the lactic acid bacteria culture medium.

TABLE 1

| | Comparative | | | | | | | | | | |
|---|---|---|---|---|---|---|---|---|---|---|---|
| Category | Example 1 | Example 1 | Example 2 | Example 3 | Example 4 | Example 5 | Example 6 | Example 7 | Example 8 | Example 9 | Example 10 |
| Dilution | 0 | 10 | 14 | 20 | 33 | 50 | 100 | 200 | 300 | 400 | 500 |
| rate (times) | | | | | | | | | | | |
| Amount | 0 | 0.1 | 0.07 | 0.05 | 0.03 | 0.02 | 0.01 | 0.005 | 0.0033 | 0.0025 | 0.002 |
| (parts | | | | | | | | | | | |
| by weight) | | | | | | | | | | | |

Comparative Example 1 and Examples 1 to 10 in Table 1 were diluted 100 times and 200 times with distilled water (pH 7), respectively, as shown in the following Table 2.

TABLE 2

| | Amount of citric acid | Dilution rate of |
|---|---|---|
| Category | (parts by weight) | distilled water (times) |
| Comparative Example 2 | 0 | 100 |
| Example 11 | 0.1 | 100 |
| Example 12 | 0.07 | 100 |
| Example 13 | 0.05 | 100 |
| Example 14 | 0.03 | 100 |
| Example 15 | 0.02 | 100 |
| Example 16 | 0.01 | 100 |
| Example 17 | 0.005 | 100 |
| Example 18 | 0.0033 | 100 |
| Example 19 | 0.0025 | 100 |
| Example 20 | 0.002 | 100 |
| Comparative Example 3 | 0 | 200 |
| Example 21 | 0.1 | 200 |
| Example 22 | 0.07 | 200 |
| Example 23 | 0.05 | 200 |
| Example 24 | 0.03 | 200 |
| Example 25 | 0.02 | 200 |
| Example 26 | 0.01 | 200 |
| Example 27 | 0.005 | 200 |
| Example 28 | 0.0033 | 200 |
| Example 29 | 0.0025 | 200 |
| Example 30 | 0.002 | 200 |

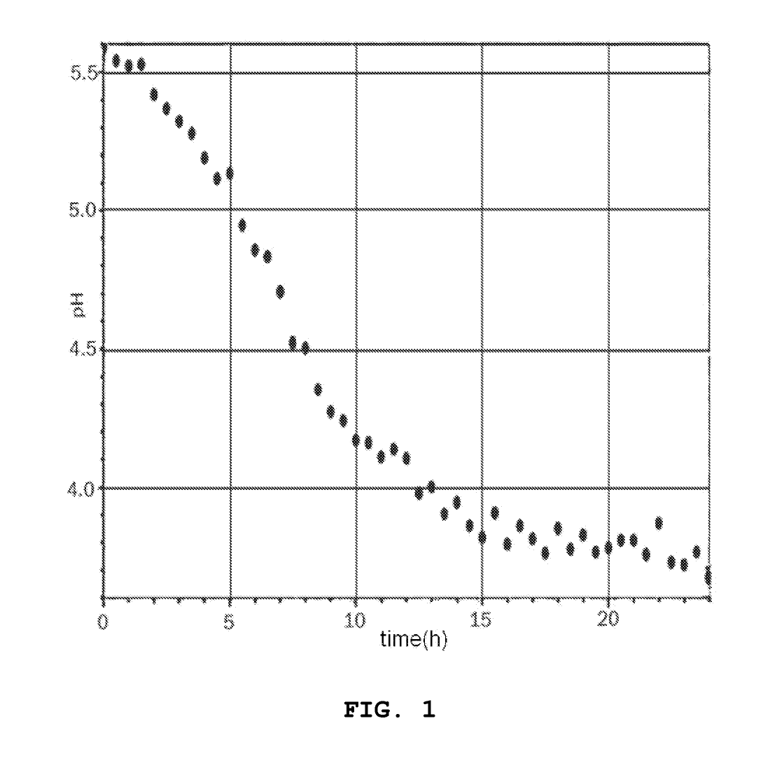

With respect to the probiotic compositions prepared according to the above Examples and Comparative Examples, pH changes of lactic acid bacteria culture medium depending on the incubation time, pH changes after addition of citric acid to the lactic acid bacteria culture medium, and pH changes after dilution of probiotic compositions were shown in FIG. 1, Table 3, and Table 4, respectively.

TABLE 3

| | Comparative | | | | | | | | | | |
|---|---|---|---|---|---|---|---|---|---|---|---|
| Category | Example 1 | Example 1 | Example 2 | Example 3 | Example 4 | Example 5 | Example 6 | Example 7 | Example 8 | Example 9 | Example 10 |
| pH | 3.67 | 2.16 | 2.25 | 2.48 | 2.68 | 2.84 | 3.04 | 3.21 | 3.3 | 3.37 | 3.45 |

TABLE 4

| Category | pH |
|---|---|
| Comparative Example 2 | 4.01 |
| Example 11 | 2.97 |
| Example 12 | 3.10 |
| Example 13 | 3.20 |
| Example 14 | 3.37 |
| Example 15 | 3.42 |
| Example 16 | 3.60 |
| Example 17 | 3.71 |
| Example 18 | 3.77 |
| Example 19 | 3.83 |
| Example 20 | 3.85 |
| Comparative Example 3 | 5.15 |
| Example 21 | 3.17 |
| Example 22 | 3.30 |
| Example 23 | 3.45 |
| Example 24 | 3.56 |
| Example 25 | 3.63 |
| Example 26 | 3.86 |
| Example 27 | 4.17 |
| Example 28 | 4.30 |
| Example 29 | 4.42 |
| Example 30 | 4.45 |

With reference to FIG. 1, the pH of the lactic acid bacteria culture medium according to one Example of the present invention was found to maintain at 4 or less after the incubation time of 15 hours. With reference to Table 3, when the amount of citric acid was 0.002 to 0.1 parts by weight, the pH of probiotic compositions (Examples 1 to 10) was within the range of 2.1 to 3.5. With reference to Table 4, probiotic compositions wherein the pH of the 100 to 200 times diluted probiotic compositions with distilled water was 4 or less (Examples 11 to 26) could be obtained.

(2) Lactic Acid Addition to Lactic Acid Bacteria Culture Medium

The probiotic composition was prepared by adding lactic acid to the lactic acid bacteria culture medium. The method for preparing the lactic acid bacteria culture medium for preparation of probiotic compositions was the same as in the above method 1.1, and like the above procedure (1), lactic acid was added, and then, the lactic acid bacteria culture medium was diluted to prepare for the concentration of lactic acid to be 0.5%, 1%, and 2%, finally.

(3) Malic Acid Addition to Lactic Acid Bacteria Culture Medium

The probiotic composition was prepared by adding malic acid to the lactic acid bacteria culture medium. The method for preparing the lactic acid bacteria culture medium for preparation of probiotic compositions was the same as in the above method 1.1, and like the above procedure (1), malic acid was added, and then, the lactic acid bacteria culture medium was diluted to prepare for the concentration of malic acid to be 0.5%, 1%, and 2%, finally.

1.3 Evaluation of Sterilization Effectiveness of Probiotic for Livestock Production

To evaluate the sterilization effectiveness of the prepared probiotic composition, citric acid, malic acid, or lactic acid was added to the lactic acid bacteria culture medium, respectively, and after dilution, the lactic acid bacteria culture medium was treated to Salmonella, E. coli, or Listeria. Inhibitory effect was evaluated by measuring total numbers of bacteria over time, and the number of bacteria was expressed as log₁₀ CFU/ml.

(1) Evaluation of Inhibitory Effectiveness on Salmonella

Standard strains Salmonella typhimurium ATCC 19585 and Salmonella typhimurium ATCC 43174 were purchased from American Type culture collection, and were incubated in Rappaport-Vassiliadis Soya Peptone Broth (CM0866, Oxoid, the United Kingdom) in a bacterial incubator at 37° C. for 24 hours. Total number of bacteria was measured by diluting strains at stationary phase to 6 to 7 log(cfu/mL).

1) Effect of Citric Acid (CA)-Added Probiotic

Salmonella bacteria in which Salmonella typhimurium ATCC 19585 and Salmonella typhimurium ATCC 43174 were mixed at a ratio of 1:1 and the probiotic compositions in Examples 5, 6 and Comparative Example 1 were mixed at a ratio of 1:1, respectively, and treated for 0 min, 10 min, 20 min, 30 min, and 60 min, and then, 10-fold serial dilution was performed, and bacteria were smeared onto MacConkey Agar (CM0115, Oxoid, the United Kingdom), the plate selective medium for Salmonella. 0.2% peptone water was used as a diluent, and the smeared plate medium was incubated in a 37° C. bacterial incubator for 24 hours, and the number of white single colony was counted. Evaluation was carried out with the average of triplicate.

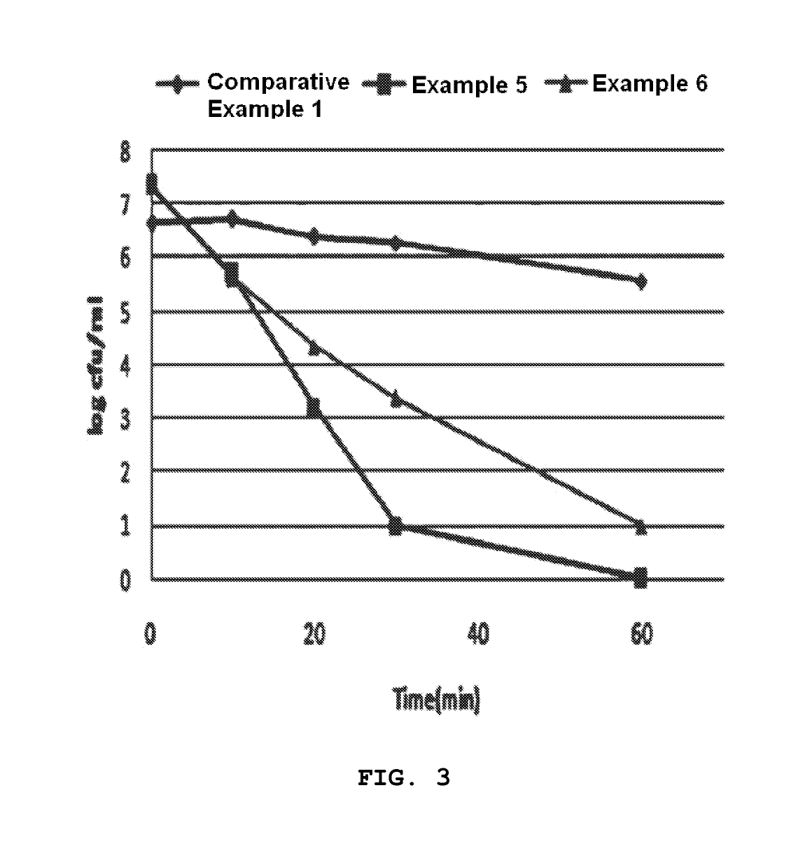

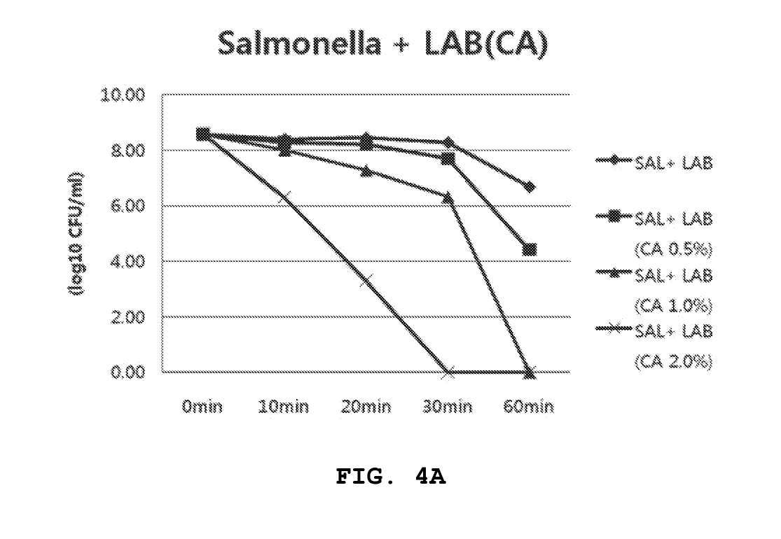

With reference to FIG. 3, even after 60 min of inoculation, while a large number (5% or more) of Salmonella bacteria were still alive in the Comparative Example 1 to which citric acid was not added, 99.9999% or more were sterilized in Examples 5 and 6 to which citric acid was added, and in the case of Example 5, 100% was sterilized.

Meanwhile, when the probiotic compositions according to Examples 5 and 6 were orally administered to a calf which was infected with Salmonella bacteria and had diarrhea, the diarrhea treatment effect was confirmed.

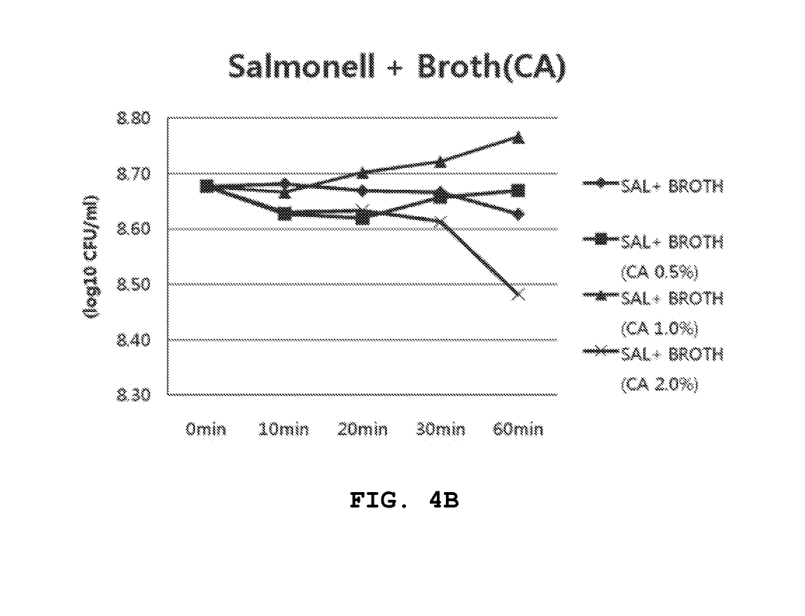

Citric acid was added to be 0.5%, 1.0%, 2.0%, respectively, and the inhibitory effect on Salmonella over time was examined. Table 5 and Table 6 show the inhibitory effects over time when the lactic acid bacteria and varying concentrations of citric acid were added to Salmonella and when only citric acid was added to Salmonella, respectively. The corresponding results were shown in FIG. 4A and FIG. 4B, respectively.

TABLE 5

| | 0 min | 10 min | 20 min | 30 min | 60 min |
|---|---|---|---|---|---|
| SAL + LAB | 8.59 | 8.40 | 8.45 | 8.28 | 6.68 |
| SAL + LAB (CA0.5%) | 8.59 | 8.28 | 8.22 | 7.70 | 4.42 |
| SAL + LAB (CA1.0%) | 8.59 | 8.02 | 7.29 | 6.33 | 0.00 |
| SAL + LAB (CA2.0%) | 8.59 | 6.31 | 3.31 | 0.00 | 0.00 |

TABLE 6

| | 0 min | 10 min | 20 min | 30 min | 60 min |
|---|---|---|---|---|---|
| SAL + BROTH | 8.68 | 8.68 | 8.67 | 8.67 | 8.63 |
| SAL + BROTH | 8.68 | 8.63 | 8.62 | 8.66 | 8.67 |
| (CA 0.5%) | | | | | |
| SAL + | 8.68 | 8.67 | 8.70 | 8.72 | 8.77 |
| BROTH (CA | | | | | |
| 1.0%) | | | | | |
| SAL + | 8.68 | 8.63 | 8.63 | 8.61 | 8.48 |
| BROTH (CA | | | | | |
| 2.0%) | | | | | |

In accordance with the above results, it was found that the probiotic of the present invention could sterilize Salmonella effectively, and had more excellent effect compared to the case in which citric acid was added alone.

2) Effect of Lactic Acid (LA)-Added Probiotic

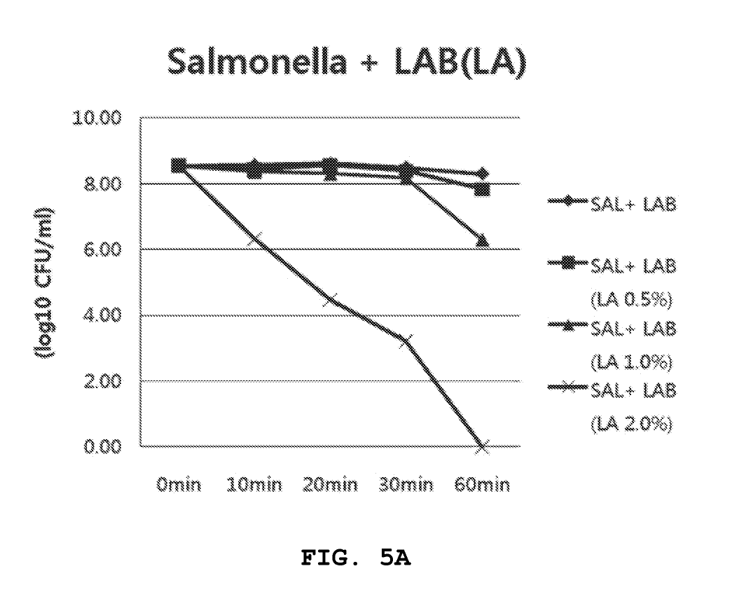

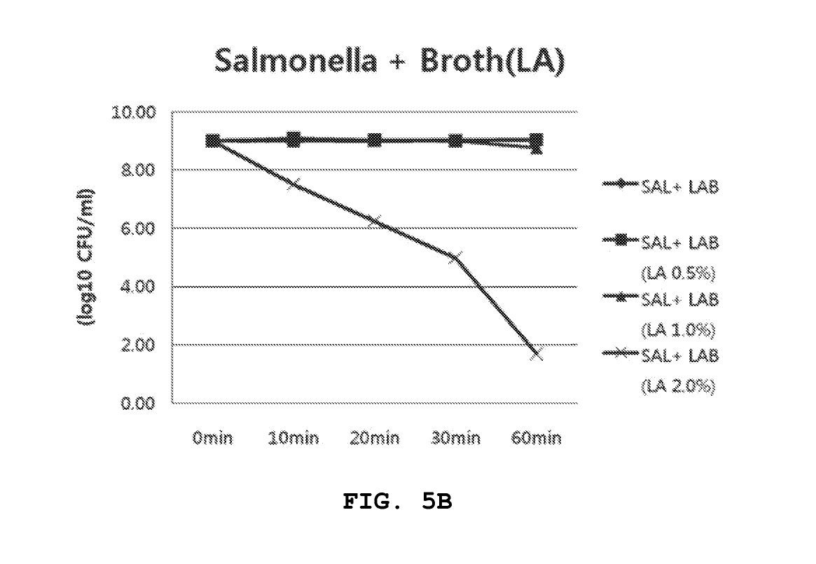

Lactic acid was added to be 0.5%, 1.0%, 2.0%, respectively, to the prepared single colony of Salmonella, and the inhibitory effect on Salmonella over time was examined. Table 7 and Table 8 show the inhibitory effects over time when the lactic acid bacteria and varying concentrations of lactic acid were added to Salmonella and when only lactic acid was added to Salmonella, respectively. The corresponding results were shown in FIG. 5A and FIG. 5B, respectively.

TABLE 7

| | 0 min | 10 min | 20 min | 30 min | 60 min |
|---|---|---|---|---|---|
| SAL + LAB | 8.55 | 8.56 | 8.61 | 8.49 | 8.30 |
| SAL + LAB | 8.55 | 8.42 | 8.56 | 8.39 | 7.83 |
| (LA0.5%) | | | | | |
| SAL + | 8.55 | 8.37 | 8.30 | 8.18 | 6.30 |
| LAB (LA1.0%) | | | | | |
| SAL + | 8.55 | 6.32 | 4.47 | 3.22 | 0.00 |
| LAB (LA2.0%) | | | | | |

TABLE 8

| | 0 min | 10 min | 20 min | 30 min | 60 min |
|---|---|---|---|---|---|
| SAL + | 8.68 | 8.68 | 8.67 | 8.67 | 8.63 |
| BROTH | | | | | |
| SAL + BROTH | 8.68 | 8.63 | 8.62 | 8.66 | 8.67 |
| (CA 0.5%) | | | | | |
| SAL + BROTH | 8.68 | 8.67 | 8.70 | 8.72 | 8.77 |
| (CA 1.0%) | | | | | |
| SAL + BROTH | 8.68 | 8.63 | 8.63 | 8.61 | 8.48 |
| (CA 2.0%) | | | | | |

In accordance with the above results, it was found that the probiotic of the present invention could sterilize Salmonella effectively, and had more excellent effect compared to the case in which lactic acid was added alone. Particularly, this difference was outstanding when 1% or more of lactic acid was added.

3) Effect of Malic Acid (MA)-Added Probiotic

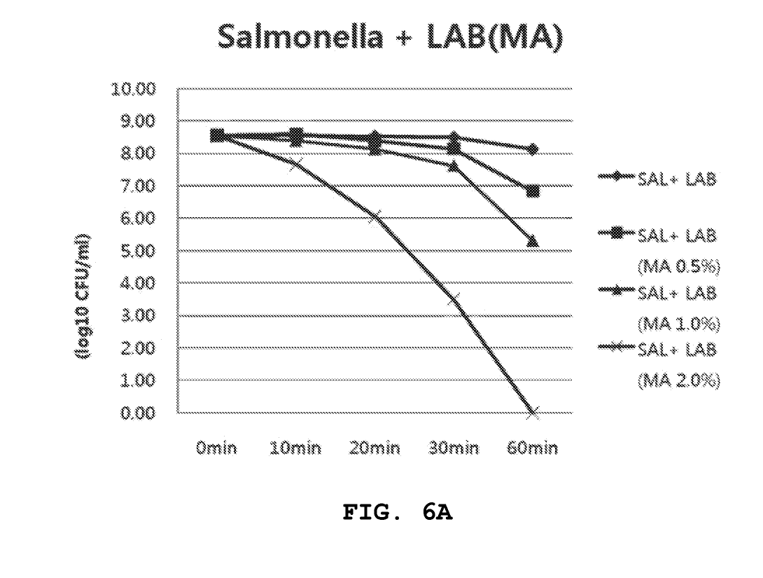

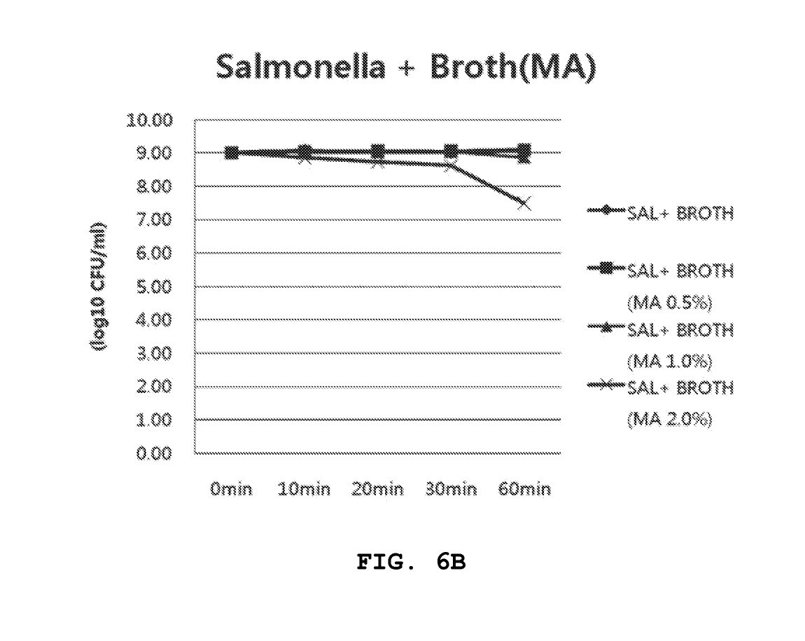

Malic acid was added to be 0.5%, 1.0%, 2.0%, respectively, to the prepared single colony of Salmonella, and the inhibitory effect on Salmonella over time was examined. Table 9 and Table 10 show the inhibitory effects over time when the lactic acid bacteria and varying concentrations of malic acid were added to Salmonella and when only malic acid was added to Salmonella, respectively. The corresponding results were shown in FIG. 6A and FIG. 6B, respectively.

TABLE 9

| | 0 min | 10 min | 20 min | 30 min | 60 min |
|---|---|---|---|---|---|
| SAL + LAB | 8.55 | 8.54 | 8.53 | 8.49 | 8.13 |
| SAL + LAB | 8.55 | 8.59 | 8.39 | 8.15 | 6.85 |
| (MA0.5%) | | | | | |
| SAL + | 8.55 | 8.40 | 8.14 | 7.63 | 5.31 |
| LAB (MA1.0%) | | | | | |
| SAL + | 8.55 | 7.67 | 6.05 | 3.51 | 0.00 |
| LAB (MA2.0%) | | | | | |

TABLE 10

| | 0 min | 10 min | 20 min | 30 min | 60 min |
|---|---|---|---|---|---|
| SAL + | 9.02 | 9.07 | 9.03 | 9.03 | 9.07 |
| BROTH | | | | | |
| SAL + | 9.02 | 9.04 | 9.05 | 9.04 | 9.09 |
| BROTH | | | | | |
| (MA 0.5%) | | | | | |
| SAL + | 9.02 | 9.03 | 9.04 | 9.04 | 8.88 |
| BROTH (MA | | | | | |
| 1.0%) | | | | | |
| SAL + | 9.02 | 8.88 | 8.73 | 8.63 | 7.50 |
| BROTH (MA | | | | | |
| 2.0%) | | | | | |

In accordance with the above results, it was found that the probiotic of the present invention could sterilize Salmonella effectively, and had more excellent effect compared to the case in which malic acid was added alone. Particularly, this difference was outstanding as the concentration of malic acid added increases, and when 2% of malic acid was added, almost all the bacteria could be removed within 1 hour, and this suggested that the probiotic of the present invention had a very excellent effect.

(2) Evaluation of Inhibitory Effectiveness on E. Coli

Standard strain E. coli and the probiotic composition of the present invention were mixed at a ratio of 1:1, respectively, and treated for 0 min, 10 min, 20 min, 30 min, and 60 min, and then, 10-fold serial dilution was performed, and bacteria were plated. 0.2% peptone water was used as a diluent, and the smeared plate medium was incubated in a 37° C. bacterial incubator for 24 hours, and the number of white single colony was counted. Evaluation was carried out with the average of triplicate.

1) Effect of Citric Acid (CA)-Added Probiotic

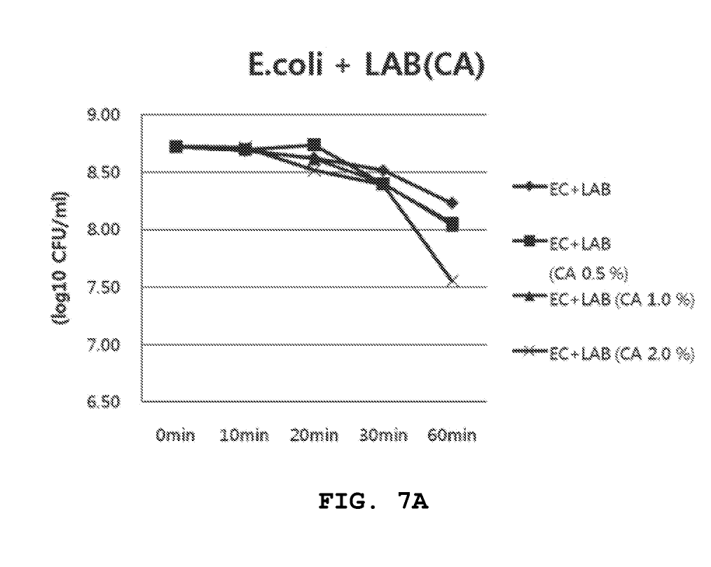

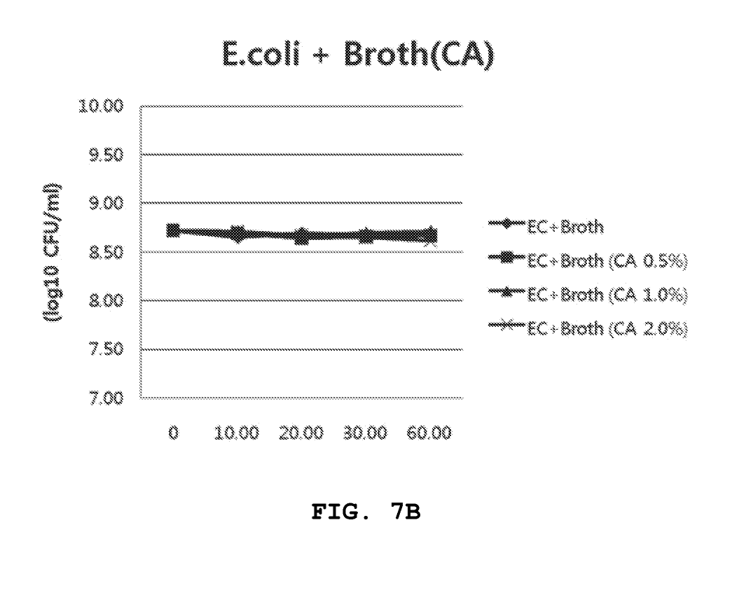

Citric acid (CA) was added to be 0.5%, 1.0%, 2.0%, respectively, and the inhibitory effect on E. coli over time was examined. Table 11 and Table 12 show the inhibitory effects over time when the lactic acid bacteria and varying concentrations of citric acid were added to E. coli and when only citric acid was added to E. coli, respectively. The corresponding results were shown in FIG. 7A and FIG. 7B, respectively.

TABLE 11

| | 0 min | 10 min | 20 min | 30 min | 60 min |
|---|---|---|---|---|---|
| EC + LAB | 8.72 | 8.69 | 8.62 | 8.51 | 8.23 |
| EC + LAB (CA | 8.72 | 8.70 | 8.74 | 8.40 | 8.05 |
| 0.5%) | | | | | |
| EC + LAB (CA | 8.72 | 8.70 | 8.61 | 8.40 | 8.03 |
| 1.0%) | | | | | |
| EC + LAB (CA 2.0%) | 8.72 | 8.72 | 8.51 | 8.39 | 7.56 |

TABLE 12

| | 0 min | 10 min | 20 min | 30 min | 60 min |
|---|---|---|---|---|---|
| EC + Broth | 8.72 | 8.65 | 8.69 | 8.68 | 8.68 |
| EC + Broth (CA | 8.72 | 8.70 | 8.64 | 8.66 | 8.67 |
| 0.5%) | | | | | |
| EC + Broth (CA | 8.72 | 8.71 | 8.65 | 8.70 | 8.71 |
| 1.0%) | | | | | |
| EC + Broth (CA | 8.72 | 8.71 | 8.67 | 8.65 | 8.61 |
| 2.0%) | | | | | |

In accordance with the above result, the probiotic of the present invention showed an apparent sterilization effect, compared to a control group to which citric acid alone was added and in which there is no change in the number of bacteria over time.

2) Effect of Lactic Acid (LA)-Added Probiotic

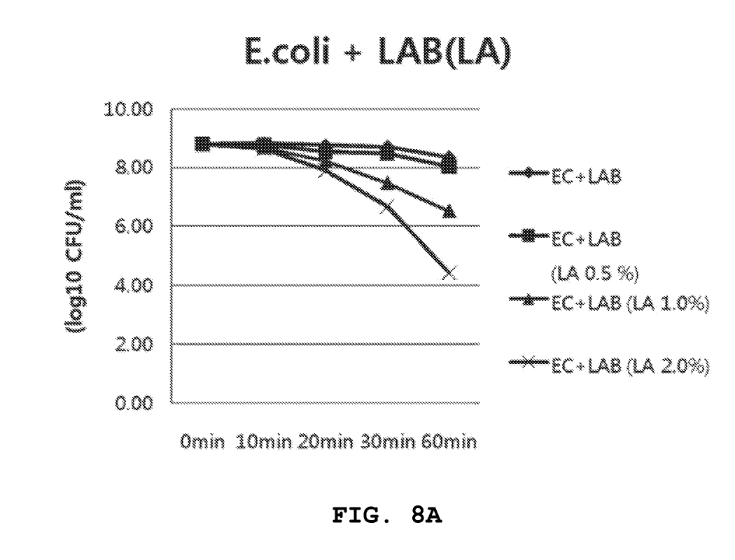

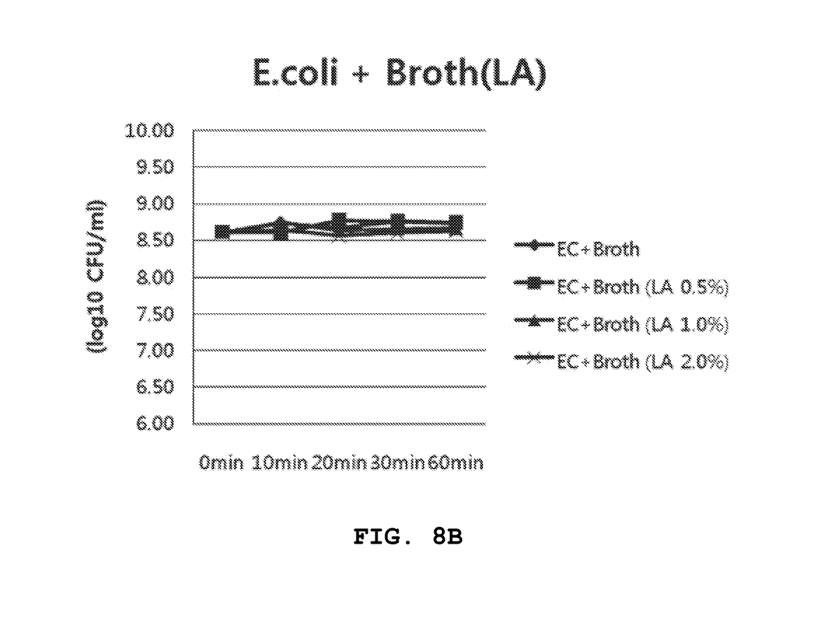

Lactic acid was added to be 0.5%, 1.0%, 2.0%, respectively, to the prepared single colony of E. coli, and the inhibitory effect on E. coli over time was examined. Table 13 and Table 14 show the inhibitory effects over time when the lactic acid bacteria and varying concentrations of lactic acid were added to E. coli and when only lactic acid was added to E. coli, respectively. The corresponding results were shown in FIG. 8A and FIG. 8B, respectively.

TABLE 13

| | 0 min | 10 min | 20 min | 30 min | 60 min |
|---|---|---|---|---|---|
| EC + LAB | 8.80 | 8.82 | 8.76 | 8.71 | 8.36 |
| EC + LAB | 8.80 | 8.78 | 8.55 | 8.47 | 8.03 |
| LA 0.5% | | | | | |
| EC + LAB | 8.80 | 8.68 | 8.21 | 7.48 | 6.52 |
| LA 1.0% | | | | | |
| EC + LAB | 8.80 | 8.64 | 7.90 | 6.66 | 4.43 |
| LA 2.0% | | | | | |

TABLE 14

| | 0 min | 10 min | 20 min | 30 min | 60 min |
|---|---|---|---|---|---|
| EC + Broth | 8.61 | 8.74 | 8.67 | 8.76 | 8.73 |
| EC + Broth | 8.61 | 8.61 | 8.78 | 8.76 | 8.74 |
| (LA 0.5%) | | | | | |
| EC + Broth | 8.61 | 8.74 | 8.63 | 8.66 | 8.66 |
| (LA 1.0%) | | | | | |
| EC + Broth | 8.61 | 8.64 | 8.56 | 8.60 | 8.63 |
| (LA 2.0%) | | | | | |

As shown in FIG. 8, the probiotic of the present invention showed an apparent sterilization effect, compared to a control group to which citric acid alone was added and in which there is no change in the number of bacteria over time.

3) Effect of Malic Acid (MA)-Added Probiotic

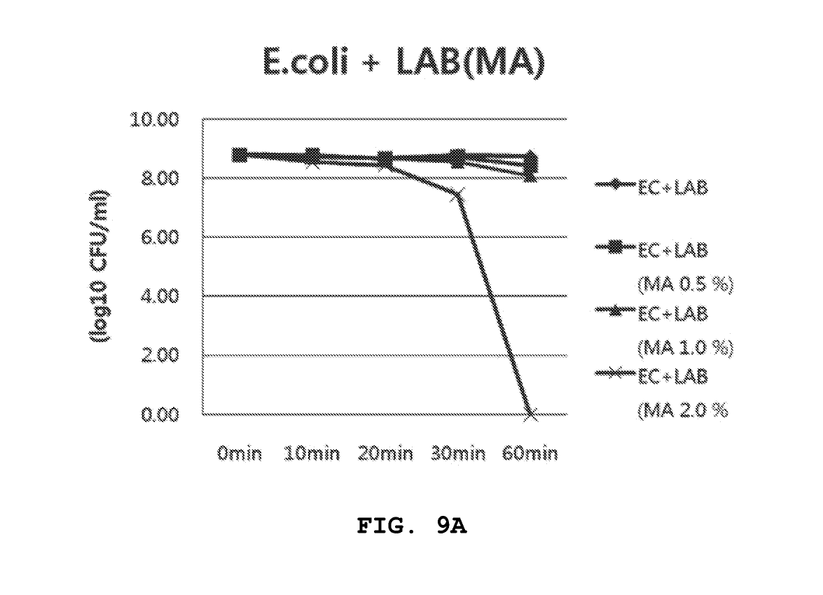

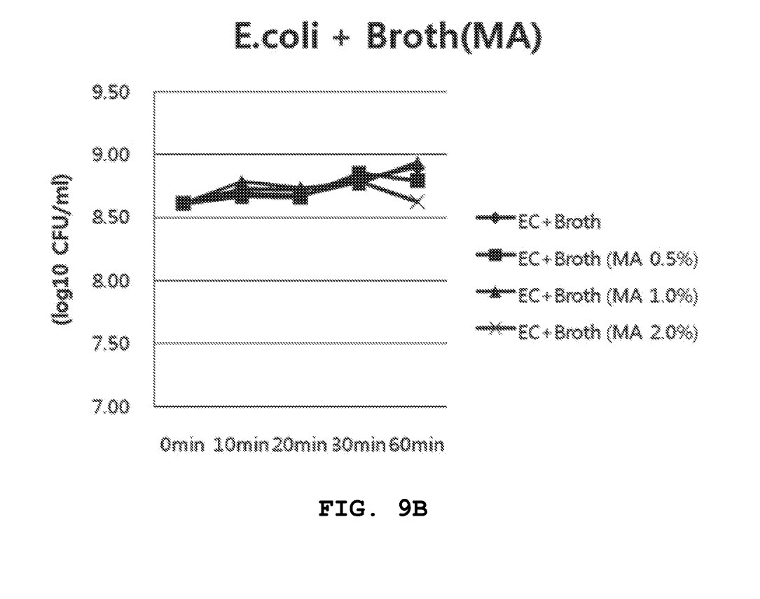

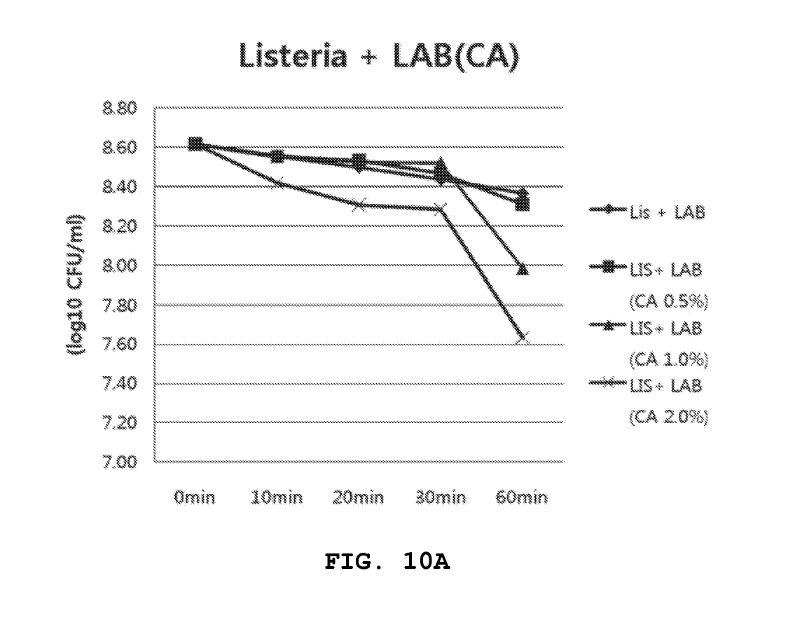

Malic acid was added to be 0.5%, 1.0%, 2.0%, respectively, to the prepared single colony of E. coli, and the inhibitory effect on E. coli over time was examined. Table 15 and Table 16 show the inhibitory effects over time when the lactic acid bacteria and varying concentrations of malic acid were added to E. coli and when only malic acid was added to E. coli, respectively. The corresponding results were shown in FIG. 9A and FIG. 9B, respectively.

TABLE 15

| | 0 min | 10 min | 20 min | 30 min | 60 min |
|---|---|---|---|---|---|
| EC + LAB | 8.80 | 8.70 | 8.67 | 8.77 | 8.74 |
| EC + LAB | 8.80 | 8.78 | 8.65 | 8.71 | 8.43 |
| MA 0.5% | | | | | |
| EC + LAB | 8.80 | 8.71 | 8.67 | 8.56 | 8.09 |
| MA 1.0% | | | | | |
| EC + LAB | 8.80 | 8.56 | 8.44 | 7.44 | 0.00 |
| MA + 2.0% | | | | | |

TABLE 16

| | 0 min | 10 min | 20 min | 30 min | 60 min |
|---|---|---|---|---|---|
| EC + Broth | 8.61 | 8.73 | 8.72 | 8.80 | 8.90 |
| EC + Broth | 8.61 | 8.67 | 8.66 | 8.86 | 8.79 |
| (MA 0.5%) | | | | | |
| EC + Broth | 8.61 | 8.78 | 8.74 | 8.77 | 8.94 |
| (MA 1.0%) | | | | | |
| EC + Broth | 8.61 | 8.69 | 8.68 | 8.79 | 8.63 |
| (MA 2.0%) | | | | | |

When 2% malic acid was added to the probiotic, the probiotic exhibited about 99.999% of sterilization effect after 60 minutes, and thus, it was confirmed that the malic acid-added probiotic of the present invention could sterilize effectively E. coli.

(3) Evaluation of Inhibitory Effectiveness on Listeria

Standard strain Listeria and the probiotic composition of the present invention were mixed at a ratio of 1:1, respectively, and treated for 0 min, 10 min, 20 min, 30 min, and 60 min, and then, 10-fold serial dilution was performed, and bacteria were plated. 0.2% peptone water was used as a diluent, and the smeared plate medium was incubated in a 37° C. bacterial incubator for 24 hours, and the number of white single colony was counted. Evaluation was carried out with the average of triplicate.

1) Effect of Citric Acid (CA)-Added Probiotic

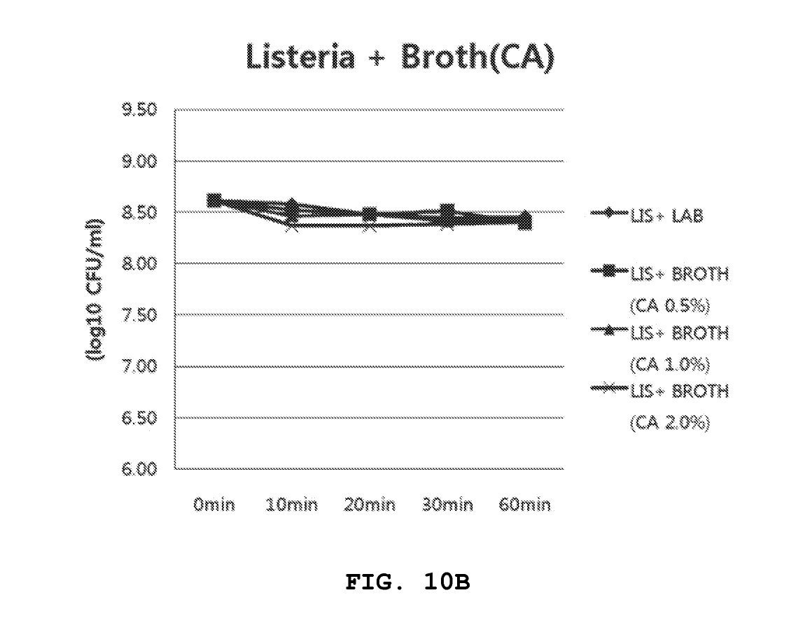

Citric acid (CA) was added to be 0.5%, 1.0%, 2.0%, respectively, and the inhibitory effect on Listeria over time was examined. Table 17 and Table 18 show the inhibitory effects over time when the lactic acid bacteria and varying concentrations of citric acid were added to Listeria and when only citric acid was added to Listeria, respectively. The corresponding results were shown in FIG. 10A and FIG. 10B, respectively.

TABLE 17

| | 0 min | 10 min | 20 min | 30 min | 60 min |
|---|---|---|---|---|---|
| Lis + LAB | 8.62 | 8.55 | 8.50 | 8.44 | 8.37 |
| LIS + LAB (CA 0.5%) | 8.62 | 8.55 | 8.53 | 8.47 | 8.31 |
| LIS + LAB (CA 1.0%) | 8.62 | 8.56 | 8.52 | 8.52 | 7.98 |
| LIS + LAB (CA 2.0%) | 8.62 | 8.42 | 8.31 | 8.29 | 7.63 |

TABLE 18

| | 0 min | 10 min | 20 min | 30 min | 60 min |
|---|---|---|---|---|---|
| LIS + LAB | 8.62 | 8.58 | 8.48 | 8.45 | 8.46 |
| LIS + BROTH | 8.62 | 8.52 | 8.48 | 8.51 | 8.39 |
| (CA 0.5%) | | | | | |
| LIS + | 8.62 | 8.47 | 8.48 | 8.41 | 8.42 |
| BROTH (CA | | | | | |
| 1.0%) | | | | | |
| LIS + | 8.62 | 8.37 | 8.37 | 8.38 | 8.40 |
| BROTH (CA | | | | | |
| 2.0%) | | | | | |

In accordance with the above result, the probiotic of the present invention showed an apparent sterilization effect, compared to a control group to which citric acid alone was added and in which there is no change in the number of bacteria over time.

2) Effect of Lactic Acid (LA)-Added Probiotic

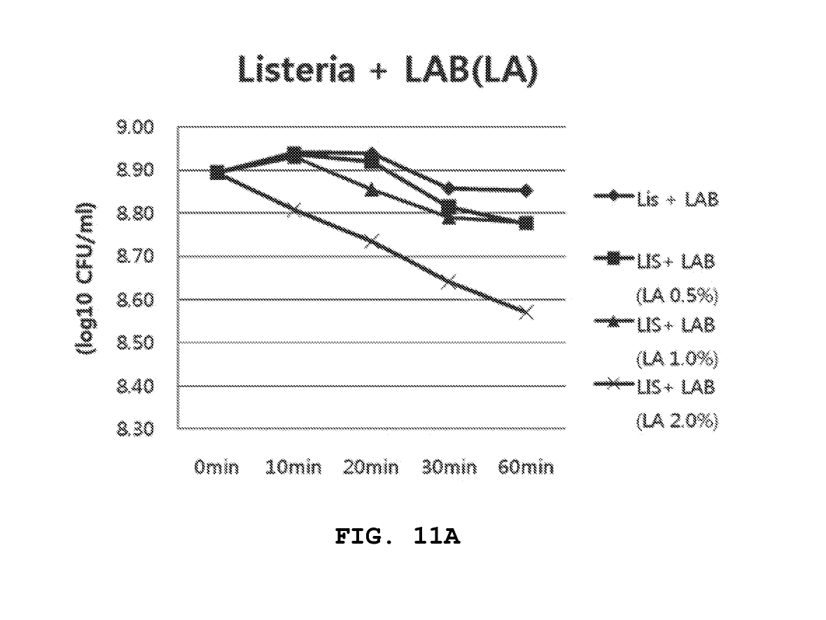

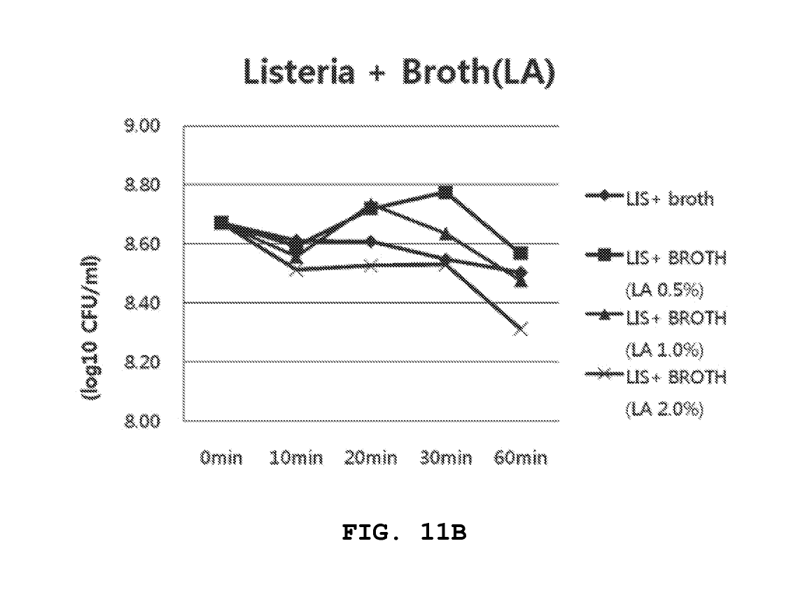

Lactic acid was added to be 0.5%, 1.0%, 2.0%, respectively, to the prepared single colony of Listeria, and the inhibitory effect on Listeria over time was examined. Table 19 and Table 20 show the inhibitory effects over time when the lactic acid bacteria and varying concentrations of lactic acid were added to Listeria and when only lactic acid was added to Listeria, respectively. The corresponding results were shown in FIG. 11A and FIG. 11B, respectively.

TABLE 19

| | 0 min | 10 min | 20 min | 30 min | 60 min |
|---|---|---|---|---|---|
| Lis + LAB | 8.89 | 8.94 | 8.94 | 8.86 | 8.85 |
| LIS + LAB | 8.89 | 8.94 | 8.92 | 8.81 | 8.78 |
| (LA 0.5%) | | | | | |
| LIS + | 8.89 | 8.93 | 8.86 | 8.79 | 8.78 |
| LAB | | | | | |
| (LA 1.0%) | | | | | |
| LIS + | 8.89 | 8.81 | 8.73 | 8.64 | 8.57 |
| LAB | | | | | |
| (LA 2.0%) | | | | | |

TABLE 20

| | 0 min | 10 min | 20 min | 30 min | 60 min |
|---|---|---|---|---|---|
| LIS + broth | 8.67 | 8.61 | 8.61 | 8.55 | 8.50 |
| LIS + | 8.67 | 8.59 | 8.72 | 8.77 | 8.57 |
| BROTH | | | | | |
| (LA 0.5%) | | | | | |
| LIS + | 8.67 | 8.56 | 8.73 | 8.64 | 8.48 |
| BROTH (LA | | | | | |
| 1.0%) | | | | | |
| LIS + | 8.67 | 8.51 | 8.53 | 8.53 | 8.31 |
| BROTH (LA | | | | | |
| 2.0%) | | | | | |

As shown in FIG. 11A, the probiotic of the present invention showed a continuous sterilization effect over time, compared to a control group to which citric acid alone was added.

3) Effect of Malic Acid (MA)-Added Probiotic

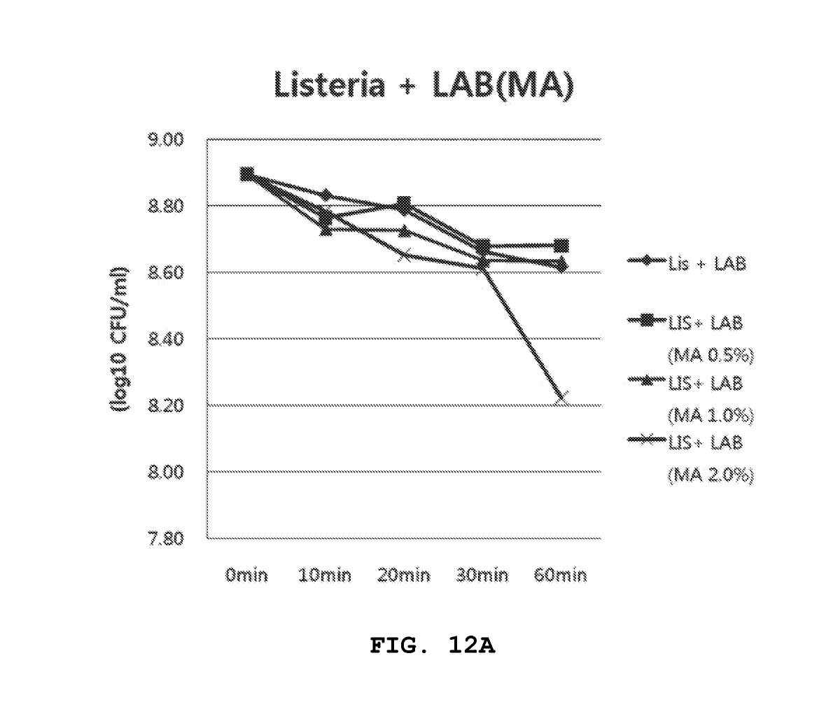

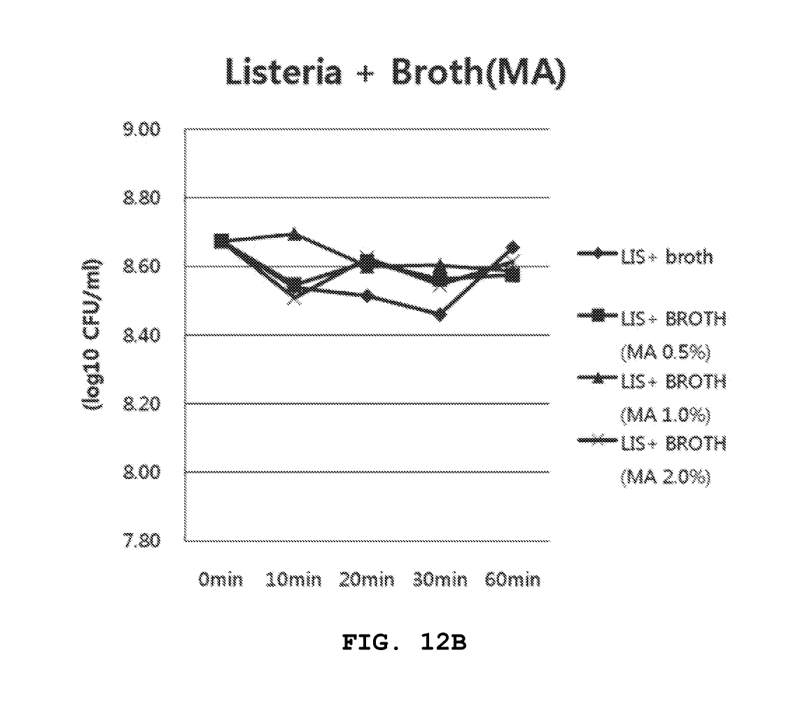

Malic acid was added to be 0.5%, 1.0%, 2.0%, respectively, to the prepared single colony of Listeria, and the inhibitory effect on Listeria over time was examined. Table 21 and Table 22 show the inhibitory effects over time when the lactic acid bacteria and varying concentrations of malic acid were added to Listeria and when only malic acid was added to Listeria, respectively. The corresponding results were shown in FIG. 12A and FIG. 12B, respectively.

TABLE 21

| | 0 min | 10 min | 20 min | 30 min | 60 min |
|---|---|---|---|---|---|
| Lis + LAB | 8.89 | 8.83 | 8.79 | 8.66 | 8.62 |
| LIS + LAB | 8.89 | 8.76 | 8.81 | 8.68 | 8.68 |
| (MA 0.5%) | | | | | |
| LIS + | 8.89 | 8.73 | 8.73 | 8.64 | 8.63 |
| LAB (MA 1.0%) | | | | | |
| LIS + | 8.89 | 8.78 | 8.65 | 8.61 | 8.22 |
| LAB (MA 2.0%) | | | | | |

TABLE 22

| | 0 min | 10 min | 20 min | 30 min | 60 min |
|---|---|---|---|---|---|
| LIS + broth | 8.67 | 8.54 | 8.51 | 8.46 | 8.65 |
| LIS + | 8.67 | 8.55 | 8.61 | 8.56 | 8.58 |
| BROTH | | | | | |
| (MA 0.5%) | | | | | |
| LIS + | 8.67 | 8.69 | 8.60 | 8.60 | 8.59 |
| BROTH(MA | | | | | |
| 1.0%) | | | | | |
| LIS + | 8.67 | 8.51 | 8.62 | 8.55 | 8.61 |
| BROTH(MA | | | | | |
| 2.0%) | | | | | |

Compared to a control group, the malic acid-added probiotic of the present invention could reduce the number of Listeria, and in particular, when 2% malic acid was added, the most effective sterilization effect was confirmed.

2. Evaluation of Virucidal Effectiveness of Probiotic for Livestock Production

The virucidal effect of the probiotic of the present invention in which the organic acid was added to the lactic acid bacteria culture medium was examined.

2.1 Virucidal Effectiveness on Avian Influenza Virus

(1) Experimental Preparation

Avian influenza virus AIV0028 (H9N2) was used for the virus of the present invention. The probiotic was prepared by adding citric acid to be in a concentration of 2% to the prepared lactic acid bacteria culture medium, and was dissolved to prepare a disinfecting liquid, and peptone water was used for a control diluent.

0.305 g of CaCl₂ and 0.139 g (w/v) of MgCl₂.6H₂O were added to 1 L of distilled water to prepare hard water, and fetal bovine serum was added to be 5% to the prepared hard water to prepare an organic matter diluent. All the diluents, including distilled water, and disinfectants, and avian influenza virus were kept at refrigerator temperatures, and tests were also carried out at refrigerator temperatures.

Disinfection effects on virus were measured for all the different kinds of diluents (distilled water, hard water, the organic matter), and effects of the diluents were measured at dilution rates of 1/50, 1/100, 1/150, and 1/200. Avian influenza virus was diluted 1/10 with each diluent to use.

1 mL of avian influenza virus diluted 1/10 with each diluent and 1 mL of the disinfectant diluted at different dilution rates were mixed and allowed to react at 4° C. for 30 minutes. Distilled water, hard water, and the organic matter were treated with the same method, and lactic acid bacteria were removed from all the experimental groups by filtering mixtures through 0.45 nm filter papers right after treatment. The disinfectant treatment-completed virus material was mixed 1:1 (equivalently) with a cell culture medium containing 50% fetal bovine serum (antibiotics and sodium bicarbonate were added) to neutralize the disinfectant. Directly after neutralization, the mixture was diluted 1/10 with a serum-free medium (antibiotics and sodium bicarbonate were added) and kept on ice to dilute the disinfection effect which may be continued after termination of treatment time, which was considered as 10⁻¹. Subsequently, decimal dilution was carried out with a serum-free medium.

The scope of inoculation was carried out 10⁻¹ to 10⁻⁶ for a disinfectant-treated virus, and 10⁻⁴ to 10⁻⁷ for a control virus.

0.1 mL of the virus was inoculated through an allantoic cavity of ten to twelve-day old embryo, and 4 to 5 days later, some of the culture medium was collected from each well and a plate HA test was carried out.

(2) Dilution Rate Effect

The probiotic containing 2% citric acid of the present invention was prepared to use as the disinfectant as in the above description, and showed the effect as in Table 23. To compare with the disinfection effect of the case treated with citric acid only, titers of avian influenza virus were measured with the varying concentrations from 2% to 8% of citric acid. The result was shown in Table 24.

TABLE 23

| | | | | | | Final |
|---|---|---|---|---|---|---|
| | | Titer of each test (log10) | | | | Effective |
| | Disinfectant | Probiotic Disinfectant | | | | Dilution |
| Diluent | Dilution Rate | 1st | 2nd | 3rd | 4th | Rate |
| Soft Water | 1:50 | 0.50 | 0.50 | 0.50 | 0.50 | 50 times |
| (Distilled | 1:100 | 4.50 | 4.00 | 1.25 | 1.75 | |
| Water) | 1:150 | 4.50 | — | — | 3.75 | |
| | 1:200 | 5.25 | — | — | 4.00 | |
| | Titer of | 5.50 | 5.88 | 4.50 | 6.00 | |
| | Pathogen | | | | | |
| | Control | | | | | |
| | Effective | 50 times | 50 times | 50 times | 100 times | |
| | Dilution Rate | | | | | |
| Hard | 1:25 | — | 0.50 | | | 50 times |
| Water | 1:50 | 0.75 | 0.50 | | | |
| | 1:100 | 2.50 | 1.50 | | | |
| | 1:150 | — | — | | | |
| | Titer of | 5.88 | 5.83 | | | |
| | Pathogen | | | | | |
| | Control | | | | | |
| | Effective | 50 times | 50 times | | | |
| | Dilution Rate | | | | | |
| Organic | 1:25 | — | 1.75 | | | 25 times |
| Water | 1:50 | 0.75 | 2.50 | | | |
| | 1:100 | 3.00 | 3.25 | | | |
| | Titer of | 4.75 | 5.83 | | | |
| | Pathogen | | | | | |
| | Control | | | | | |
| | Effective | 50 times | 25 times | | | |
| | Dilution Rate | | | | | |

TABLE 24

| Citric Acid | | | Avian Influenza |
|---|---|---|---|
| Concentration | Diluent | Dilution Rate | Titer (log 10) |
| 2% | Soft Water | 1:12.5 | 1.25 |
| 2% | (Distilled Water) | 1:25 | 2.00 |
| 4% | | 1:50 | 2.00 |
| 6% | | 1:50 | 2.00 |
| 8% | | 1:50 | 0.75 |

| | |
|---|---|
| Titer of Pathogen Control | 5.00 |

The probiotic of the present invention in which 2% citric acid was added to the lactic acid bacteria showed the disinfection effectiveness similar to the case in which 8% citric acid was added against avian influenza virus. In particular, when looking at the effective dilution concentration, the disinfection effectiveness was up to 50 to 100 times in distilled water, 50 times in hard water, and 25 to 50 times in the organic matter. From the results, it was found that the probiotic of the present invention could exhibit virucidal effect effectively with the addition of low concentration of citric acid.

2.2 Virucidal Effectiveness on Foot-and-Mouth Disease Virus (FMDV)

The virucidal effect of the probiotic of the present invention on the foot-and-mouth disease was examined by using A22 Iraq 106 TCID50 as the foot-and-mouth disease virus.

The probiotic used as the virus disinfectant was prepared by the same method as in the above 2.1, and other hard water and the organic matter were prepared by the same method. The experimental result for virucidal effect of the probiotic depending on the dilution rate was shown in Table 25.

TABLE 25

| | Dilution | Log value | | | |
|---|---|---|---|---|---|
| Category | Rate | Repeat 1 | Repeat 2 | Repeat 3 | Note |
| Disinfectant | 10 times | — | — | — | No CPE |
| (Diluted with | 20 times | 1.5 | — | 0.9 | |
| Hard | 50 times | 4.5 | 4.3 | 4.8 | |
| Water) + | | | | | |
| FMDV | | | | | |
| Disinfectant | 10 times | — | — | — | No CPE |
| (Diluted with | 20 times | 1.1 | — | 0.9 | |
| Organic | 50 times | 4.7 | 4.7 | 4.9 | |
| Matter- | | | | | |
| containing | | | | | |
| Hard | | | | | |
| Water) + | | | | | |
| FMDV | | | | | |
| Hard | | 5.1 | 5.5 | 5.5 | |
| Water + | | | | | |
| FMDV | | | | | |
| Organic | | 5.2 | 5.3 | 5.4 | |
| Matter- | | | | | |
| containing | | | | | |
| Hard | | | | | |
| Water | | | | | |
| (FBS5%) + | | | | | |
| FMDV | | | | | |

As determined from the above Table, although the virucidal effect was reduced, it appeared even in the 50 times dilution. When dilution was carried out up to 20 times, the effective virucidal effect was observed. Therefore, it was confirmed that the probiotic composition of the present invention can be used as an effective disinfectant against viruses.

3. Evaluation of Preservative Effect Improvement of Probiotic for Livestock Production

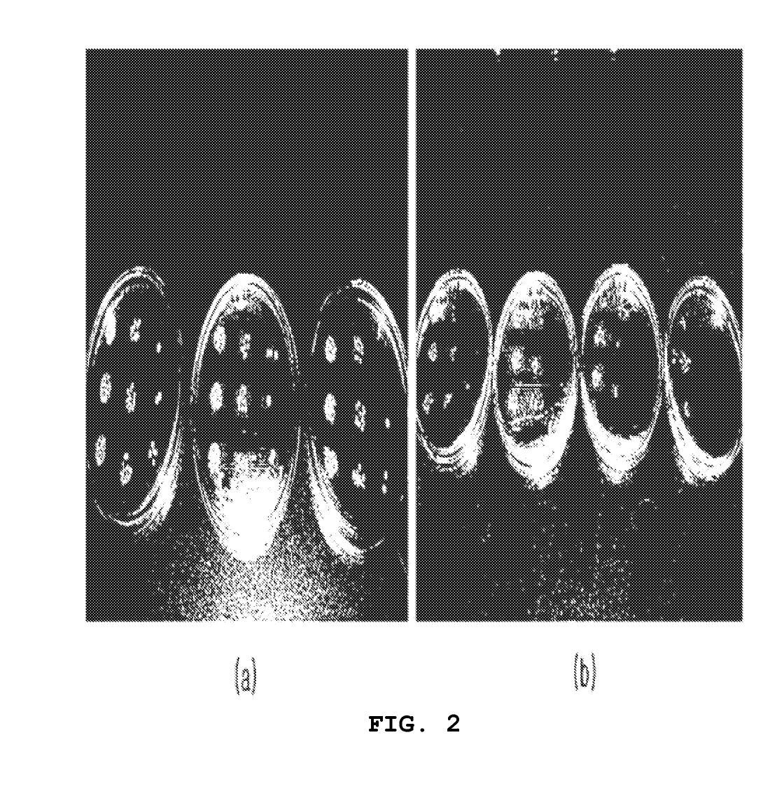

After addition of citric acid to the lactic acid bacteria culture medium, the number of lactic acid bacteria was evaluated by the following method, and the result was shown in the following Table 26. In addition, FIG. 2 is photographs showing the results of incubation tests following the examination for the number of lactic acid bacteria of Comparative Example 1 and Examples 1 to 6 in Table 1, and FIG. 2A is a photograph showing the results of Comparative Example 1, Examples 6 and 5 from the left, and FIG. 2B is a photograph showing the results of Examples 4 to 1 from the left.

The number of lactic acid bacteria (CFU/mL) was examined on MRS agar using the probiotic compositions in Comparative Example 1, Examples 1 to 7, and Example 10.

TABLE 26

| | | Comparative | | | | | | | | |
|---|---|---|---|---|---|---|---|---|---|---|
| Category | | Example 1 | Example 1 | Example 2 | Example 3 | Example 4 | Example 5 | Example 6 | Example 7 | Example 10 |
| Number | 1^{st} | 1.9 × 10⁸ | 6.0 × 10⁶ | 4.0 × 10⁷ | 5.0 × 10⁷ | 4.0 × 10⁷ | 2.0 × 10⁸ | 1.8 × 10⁸ | 1.5 × 10⁸ | 2.2 × 10⁸ |
| of | 2^{nd} | 2.3 × 10⁸ | 1.1 × 10⁷ | 2.0 × 10⁷ | 6.0 × 10⁷ | 5.0 × 10⁷ | 1.6 × 10⁸ | 1.5 × 10⁸ | 1.8 × 10⁸ | 2.3 × 10⁸ |
| lactic | 3^{rd} | 2.2 × 10⁸ | 4.0 × 10⁶ | 2.0 × 10⁷ | 4.0 × 10⁷ | 8.0 × 10⁷ | 1.3 × 10⁸ | 2.4 × 10⁸ | 2.7 × 10⁸ | 2.4 × 10⁸ |
| acid | | | | | | | | | | |
| bacteria | | | | | | | | | | |

| | | | | | | | | | |
|---|---|---|---|---|---|---|---|---|---|
| Average | 2.1 × 10⁸ | 7.0 × 10⁶ | 2.7 × 10⁷ | 5.0 × 10⁷ | 5.7 × 10⁷ | 1.6 × 10⁸ | 1.9 × 10⁸ | 2.0 × 10⁸ | 2.3 × 10⁸ |

With reference to Table 26, the number of lactic acid bacteria in the lactic acid bacteria culture medium prepared by the culture medium for lactic acid bacteria used in the probiotic composition for livestock production according to the present invention (see Comparative Example 1) was greatly excellent compared to conventional cases, and a large number of lactic acid bacteria were alive even when the amount of citric acid was up to 0.1 parts by weight, and in particular, when the amount of citric acid was 0.002 to 0.02 parts by weight (Examples 5 to 7 and Example 10), there was little or no growth inhibition on lactic acid bacteria.

Though it was found that there was little or no growth inhibition on lactic acid bacteria within the optimal concentrations of citric acid in the probiotic of the present invention, experiments for searching an appropriate liquid stabilizer were carried out to maintain the survival of lactic acid bacteria in the probiotic for livestock production in practice. Based on the above results, 2% of citric acid addition was selected as an object of the experiment for a liquid preparation, and whether the survivability of lactic acid bacteria is improved or not was examined with different kinds of preservatives.

Each liquid stabilizer was added in an amount of 0.001 parts by weight or 0.001 parts by weight relative to 1 parts by weight of the lactic acid bacteria culture medium.

TABLE 27

| | | | Trehalose | | Glycerol | | Fructooligosaccaride | |
|---|---|---|---|---|---|---|---|---|
| | No Addition | | 0.01 | | 0.01 | | 0.01 | |
| Period | Numbers | | Numbers | | Numbers | | Numbers | |
| of | of Live | Survival | of Live | Survival | of Live | Survival | of Live | Survival |
| storage | Bacteria | Rate | Bacteria | Rate | Bacteria | Rate | Bacteria | Rate |
| (day) | (CFU/ml) | (%) | (CFU/ml) | (%) | (CFU/ml) | (%) | (CFU/ml) | (%) |
| 0 | 1.62E+09 | 100 | 1.50E+09 | 100 | 1.59E+09 | 100 | 1.67E+09 | 100 |
| 5 | 1.51E+09 | 93.2 | 1.42E+09 | 94.7 | 1.44E+09 | 90.6 | 1.59E+09 | 95.2 |
| 7 | 1.50E+09 | 92.6 | 1.41E+09 | 94 | 1.20E+09 | 75.5 | 1.69E+09 | 101.2 |
| 12 | 6.13E+08 | 37.8 | 3.60E+08 | 24 | 3.37E+08 | 21.2 | 4.37E+08 | 26.2 |
| 14 | 5.00E+07 | 3.1 | 8.67E+07 | 5.8 | 8.13E+08 | 38.6 | 3.03E+08 | 18.1 |

According to the result of Table 27, when glycerol or fructooligosaccharide was added to the probiotic composition in a liquid preparation in which 2% concentration of citric acid was added, the survival rate was about 20% to 40% after 14 days were passed. Therefore, it was confirmed that a liquid stabilizer could be additionally added, in order to extend the duration of life of lactic acid bacteria in the probiotic of the present invention, and use the probiotic effectively, and for long-term preservation, glycerol and fructooligosaccharide were found to be the most suitable liquid stabilizers.

### INDUSTRIAL APPLICABILITY

The probiotic composition for livestock production of the present invention is an eco-friendly material containing lactic acid bacteria and an organic acid, and shows complex effectiveness of a sterilizer and a disinfectant. Therefore, it can sterilize and disinfect bacteria and viruses without adding antibiotics, and can be applied usefully for livestock industry.

1. A probiotic composition for livestock production, comprising lactic acid bacteria culture medium and an organic acid, wherein the organic acid is contained in an amount from about 0.002 to 0.1 parts by weight relative to 1 parts by weight of the lactic acid bacteria culture medium.

2. The probiotic composition for livestock production as claimed in claim 1, wherein the organic acid is one organic acid selected from the group consisting of citric acid, malic acid, and lactic acid.

3. The probiotic composition for livestock production as claimed in claim 1, wherein the pH of the probiotic composition for livestock production diluted about from 10 to about 100 times is 4 or less.

4. The probiotic composition for livestock production as claimed in claim 1, wherein the probiotic composition for livestock production has sterilization effectiveness.

5. A method for preparing a probiotic composition for livestock production, comprising the steps of:
(a) preparing a lactic acid bacteria culture medium;
(b) preparing a probiotic composition of a pH of from about 2.1 to about 3.5 by adding an organic acid to (a) the lactic acid bacteria culture medium.

6. The method for preparing a probiotic composition for livestock production as claimed in claim 5, wherein (a) the lactic acid bacteria culture medium is prepared by using a culture medium comprising from about 5 to about 50% of yeast extract, from about 1 to about 10% of glucose, and from about 1 to about 10% of sucrose, performing a stationary culture at from about 34 to about 36° C. for from about 15 to about 48 hours.

7. The method for preparing a probiotic composition for livestock production as claimed in claim 5, further comprising (d) preparing a probiotic composition of a pH of 4 or less, while diluting the probiotic composition from about 10 to about 100 times.

8. The method for preparing a probiotic composition for livestock production as claimed in claim 5, wherein the probiotic composition for livestock production has sterilization effectiveness.

9. The probiotic composition for livestock production as claimed in claim 1, wherein the probiotic composition for livestock production has virucidal effectiveness.

10. The probiotic composition for livestock production as claimed in claim 9, wherein the virus is a foot-and-mouth disease virus or an avian influenza virus.

11. A virus disinfectant composition for livestock production as claimed in claim 9, wherein the probiotic composition for livestock production is diluted from about 10 times to about 50 times.

12. A method for improving a preservative effect of a probiotic composition for livestock production, wherein a liquid stabilizer is added to a probiotic composition for livestock production in which an organic acid is contained in an amount from about 0.002 to about 0.1 parts by weight relative to 1 parts by weight of a lactic acid bacteria culture medium.

13. The method for improving a preservative effect of a probiotic composition for livestock production as claimed in claim 12, wherein the liquid stabilizer is one or more stabilizers selected from the group consisting of trehalose, glycerol, fructooligosaccharide, betaine, proline, dextran, maltose, sucrose, mannitol, polyol, silica gel, aminoguanidine, and pyridoxamine.

14. The method for improving a preservative effect of a probiotic composition for livestock production as claimed in claim 13, wherein the liquid stabilizer is added in an amount from about 0.001 to about 0.1 parts by weight relative to 1 parts by weight of the lactic acid bacteria culture medium.

15. A sterilizing and disinfecting method, wherein the probiotic composition for livestock production as claimed in claim 1 is orally administered to animals.

16. A sterilizing and disinfecting method, wherein the probiotic composition for livestock production as claimed in claim 1 is sprayed onto livestock sheds.

17. The probiotic composition for livestock production as claimed in claim 1, wherein the probiotic composition for livestock production is formulated in a liquid or powder preparation.
